(19) United States
(12) Patent Application Publication
(10) Pub. No.: US 2009/0056419 A1
(43) Pub. Date: Mar. 5, 2009

(54) HIGH EFFICIENCY, LOW LOSS NO TO NO2 CATALYTIC CONVERTER

(75) Inventors: Ying-Lan CHANG, Cupertino, CA (US); Bradley N. JOHNSON, Berkeley, CA (US); Craig BRYANT, Alameda, CA (US); William MICKELSON, San Francisco, CA (US)

Correspondence Address: Weaver Austin Villeneuve & Sampson LLP, P.O. BOX 70250, OAKLAND, CA, 94612-0250, US

(73) Assignee: Nanomix, Inc., Emeryville, CA (US)

(21) Appl. No.: 12/193,353

(22) Filed: Aug. 18, 2008

Related U.S. Application Data
(63) Continuation-in-part of application No. 11/924,328, filed on Oct. 25, 2007.
(60) Provisional application No. 60/967,552, filed on Sep. 4, 2007.

(51) Int. Cl. G01N 33/00; B01J 19/00; B01J 8/06

(52) U.S. Cl. 73/31.05; 422/198; 422/211

(57) ABSTRACT

Provided herein are catalytic converters that have improved characteristics. According to various embodiments, the converters include high surface area catalyst supports conformally coated with nanoparticulate thin films of a catalyst (e.g., Pt, Pd and Rh). The films are continuous, preventing absorption of species within the converter on the catalyst support. The converters provide higher oxidation efficiency than conventional catalytic converters, in certain embodiments approaching the stoichiometric ratio for the reaction. The converters also provide minimal loss of chemical species within the converter. Also provided are novel methods of fabricating catalytic converters that involve atomic layer deposition of Pt or other catalyst on the support, as well as methods and devices for sensing NO in samples that involve catalytic conversion of NO to NO2.

### CROSS-REFERENCE TO RELATED APPLICATIONS

This application is a continuation-in-part of and claims priority pursuant to 35 USC. §120 of the following US application,
U.S. Ser. No. 11/924,328 filed Oct. 25, 2007 (published ______), entitled “Sensor Having A Thin-Film Inhibition Layer, Nitric Oxide Converter And Monitor”; which claims priority to U.S. provisional application No. 60/967,552 filed Sep. 4, 2007. Both of these are incorporated by reference herein.

### BACKGROUND

1. Field of the Invention

The present invention relates high efficiency, low loss catalytic converters, including NO to NO2 converters, and methods of their fabrication.

2. Description of Related Art

NO detection in breath is a proven marker for airway inflammation (as well as for other tissue inflammation, immune responses, and other conditions). Therefore, the ability to measure NO as an exhaled breath parameter, for example as fractional exhaled nitric oxide (FeNO), is a valuable tool for diagnosis, monitoring, and managed treatment of asthma and other disorders. See, for example, U.S. Pat. No. 6,010,459 entitled “Method and apparatus for the measurement of components of exhaled breath in humans”, which is incorporated by reference. However, medical systems for the measurement of NO suffer from generally the same limitations as capnograph devices, e.g., high cost, weight and complexity.

There are several different conventional technologies for sensing NO gas for medical breath analysis applications. In laser detection, a laser may be tuned to a frequency which is selectively absorbed by NO. A photo detector then detects the transmission of laser light through a sample column, the degree of absorption by the gas being related to NO concentration. NO may also be detected by such methods as chemiluminescence, and other optical detection methods. See, for example, U.S. Pat. No. 6,038,913 entitled “Device for determining the levels of NO in exhaled air”; US Published Application No. 2003-0134,427, entitled “Method and apparatus for determining gas concentration”, and US Published Application No. 2004-0017,570 entitled “Device and system for the quantification of breath gases”, each of which is incorporated by reference. However, each of the conventional NO detection strategies suffer limitations in equipment size, weight, cost and/or operational complexity that limit their use for a low-cost, patient-portable device.

### SUMMARY

Provided herein are improved catalytic converters that may be used in detection devices. According to various embodiments, the converters include high surface area catalyst supports conformally coated with nanoparticulate thin films of a catalyst (e.g., Pt, Pd and Rh). The films are continuous, providing for efficient use of the catalyst support surface area and for efficient use of the catalyst material. As a conformal and complete coating, the catalyst film is also preventing absorption of species on the catalyst support. The converters also provide minimal loss of chemical species within the converters. The converters provide higher oxidation efficiency than conventional catalytic converters in certain embodiments approaching the stoichiometric ratio for the reaction. Also provided are novel methods of fabricating catalytic converters that involve atomic layer deposition of Pt or other catalyst on the support, as well as methods and devices for sensing NO in samples that involve catalytic conversion of NO to NO2.

### SUMMARY OF FIGURES

FIG. 1 illustrates an embodiment of a catalytic converter having aspects of the invention, and configured (in this example) for low-loss, low-flow catalytic conversion of nitric oxide to nitrogen dioxide (NO-NO2).

FIG. 2A illustrates examples of forms of catalyst supports that may be used according to various embodiments.

FIG. 2B illustrates an embodiment of portion of a fibrous catalytic converter having an interlayer disposed between the fibrous matrix and the catalyst film.

FIG. 3 illustrates an embodiment of a catalytic converter having an electrical connection to the catalyst film.

FIG. 4 is a flow sheet illustrating certain operations in a method of fabricating a catalytic converter according to certain embodiments.

FIGS. 5A-5D are images of Pt deposited on quartz wool by a wet chemistry process.

FIGS. 5E and 5F are images of Pt deposited on quartz wool by an atomic layer deposition process.

FIG. 6 illustrates a fibrous catalytic converter matrix before and after being coated with a continuous thin film catalyst material.

FIG. 7 illustrates the operation one exemplary embodiment having aspects of the invention, and the effect for an electron donating (NH3) and electron withdrawing (NO2) species on the nanotube field-effect transistor (NTFET) device characteristic.

FIG. 8 shows the response of an exemplary NTFET embodiment having aspects of the invention and including PEI polymer recognition layer to four brief exposures of NO2 gas with different concentration.

FIG. 9 shows a plot showing the dependence of NO in exhaled breath on exhalation rate, reproduced from U.S. Pat. No. 6,733,463.

FIG. 10 shows a representative plot of the profile of fractional composition of NO in a patient's exhaled breath.

### DETAILED DESCRIPTION

One aspect of the invention relates to catalytic converters to convert NO to NO2. At room temperature and pressure in an oxygen-containing environment (e.g., air), nitric oxide (NO) gas eventually becomes nitrogen dioxide (NO2). This reaction can take hours or days. It may be sped up at elevated temperatures, but can still take minutes or hours.

There are a number of diverse industrial applications where NO is converted and/or destroyed. Generally in those applications, the ultimate goal is the removal of all NOx compounds, as these compounds are toxins and/or pollutants. In such industrial applications, NO may be converted to NO2, the NO2 may be in turn converted to N2 and O2, and typically it is irrelevant if some of the NO or NO2 is lost or destroyed, as that is the ultimate goal.

An example of a Pt catalyst used in industry to convert NO to NO2 to N2 and O2 is a typical automotive catalytic converter, and similar converters for other pollution control uses. These are generally made of a ceramic matrix with a wash coat of catalyst. A typical 3-way catalytic converter may include Pt, Rh, and Pd. The ceramic matrix in these converters adsorbs NO, thus having a net scrubbing effect on NO concentration. These converters, however, have high loss with much of the reactants or products absorbed onto the converter. While appropriate for automobiles and other applications in which the object is to clean up emissions, these converters cannot be used for quantitative analysis of a sample. In addition, current technology is geared toward high volume/flow/concentration applications.

Another method of converting NO to NO2 is to react it with ozone (O3). This method has good conversion efficiency, but requires not only the generation of ozone, but also may require the scrubbing of the residual ozone after the conversion is complete since excess ozone is needed in the reaction. The excess ozone is required from practical kinetics standpoint to achieve 100% conversion efficiency. This makes the conversion process more complicated and potentially dangerous because of the ozone.

The catalytic converters described herein include a matrix or substrate conformally coated with Pt or other catalyst material. In one example, a quartz wool is conformally coated with Pt such that the exposure of matrix is minimal. The catalyst coating is conformal and continuous at nanoscale dimensions, such that the contact of reactant or product molecules (e.g., NO and NO2) with the underlying matrix can be minimized.

Microscopically, catalyst on previous conventional converters forms “islands” with areas of the substrate exposed. These islands are not very efficient in terms of active surface area versus catalyst volume, as the inner mass of the islands are unavailable as reaction surfaces. The substrates provide catalyst support and are typically made porous to provide high surface areas. The catalyst on conventional converters thus provides the exposed reaction sites necessary to lower the reaction activation energy but also allows NO or other reactants/products to be lost via adsorption or other interaction with the substrate.

The converters described herein by contrast have a catalyst film that is microscopically continuous, with tight nanoparticle packing, such that the support matrix is not exposed to the NO2 and NO molecules. The nanoparticles provide high surface area without relying on the porosity of the substrates.

As a result the converters described herein have low loss—as low as 4%, 2% and in certain embodiments, approaching 0% of the total NOx species. Conversion of NO to NO2 efficiency is also high, e.g., at least 80%, 90%, 96%, 98% and in certain embodiments, approaching 100%.

The idea of conversion efficiency is used to capture the extent to which reaction proceeds to the right in the mass balance equation. Efficiency is the ratio of the amount of obtained reaction product, divided by the expected reaction product based on the mass balance equation.

The term efficiency as used herein refers to the amount of NO (or other reactant) converted to the desired product, in this embodiment, NO2. In this embodiment, the complete, or 100% efficient conversion of said number of moles of NO would yield the same said number of moles of NO2 with no remaining moles of NO.

As measured in the product stream: conversion efficiency=[NO2_out]/[NO_in+NO2_in], by moles.

The term loss as used herein refers to the amount of reactant or product absorbed or otherwise lost in the converter. For the NO to NO2 conversion, it is measured by [(NO)in−(NO2+NO)Out]/(NO)in. Catalytic converters that absorb the reactants and products may have high efficiency but will have high loss.

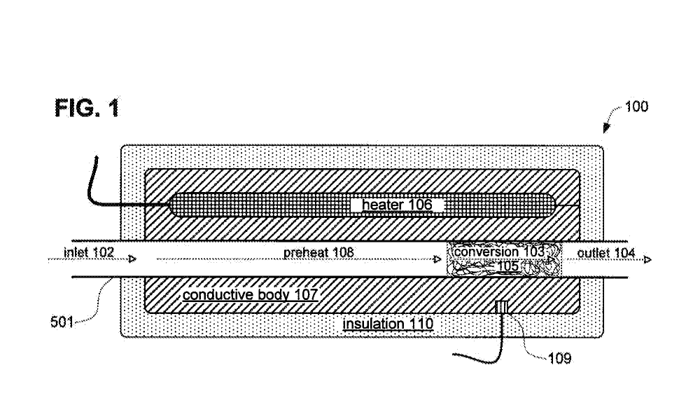

FIG. 1 illustrates an embodiment of a catalytic converter 100 having aspects of the invention, and configured (in this example) for low-loss, low-flow catalytic conversion of nitric oxide to nitrogen dioxide (NO—NO2). Converter 100 comprises a conduit 101 including, in communicating sequence: (i) an inlet portion 102 configured to receive the breath sample under an input pressure sufficient to induce flow in the conduit, a conversion portion 103, and an outlet portion 104 configured to dispense the breath sample following conversion; and a conversion material 105 disposed within the conversion portion. The conversion material 105 may be an active substance promoting conversion of NO to NO2 (e.g., a catalyst) and a carrier material or matrix configured to support the active substance in contact with the breath sample.

In this example, a catalyst such as Pt metal may be used as a catalyst to lower the energy barrier, and therefore increase the reaction rate under certain conditions. Alternative or additional catalyst materials, such as Rh, and Pd may be included. The embodiment provides that the oxidation state and physical form of the platinum metal may be optimized for conversion efficiency and stability.

The matrix is suitable to mechanically support the catalyst, binds well to Pt or other catalyst and has high surface area to provide reaction sites. In certain embodiments, a matrix material that is inert to the reactants and products, e.g., so that there is no back-conversion nor chemical or physical adsorption or other interaction of the reactants or products with the matrix. Exemplary materials include quartz, diamond, alumina, silica or glass, e.g., in the form of beads or wire, and relatively inert metals such as tungsten. As described below, in certain embodiments, the Pt or other catalyst completely covers the matrix, leaving negligible or substantially no areas of the matrix exposed to the gaseous reactants and products. In these instances, the potential interaction of the matrix material with the NO, NO2 or other gaseous components is less significant as the material does not contact the components. In certain embodiments, to support a smooth and continuous catalyst film, the matrix surface is very smooth with few defects. Surfaces having a uniform grain structure such as fused quartz are suitable for deposition of a smooth, continuous catalyst film. Other materials that form suitable surfaces for deposition include glass, alumina and bucky paper.

In certain embodiments, in addition to physically supporting the catalyst material, the matrix has one or more additional functions, e.g., as a heating element or to provide hydrophobic characteristics for operation in humid conditions. These are discussed further below.

In order to achieve a high surface area of Pt, a particular embodiment includes catalyst material (e.g., Pt particles) deposited on a carrier material, such as a wool-like fibrous substrate (e.g., quartz wool having fiber diameters ranging from about 6 to about 15 microns). It has been found that high purity, high quality quartz wool allows NO to pass without absorption at concentrations down to the ppb level. This is critical for applications that require low level detection with high accuracy.

The matrix shape is dictated by the surface area needed as well as the geometry and other particulars of the application. In certain embodiments, a fibrous structure such as quartz wool is used. Fused quartz wool may be obtained from Wale Apparatus Co., Inc., Hellertown, Pa. In another embodiment, a fibrous structure of bucky paper is used to as the catalytic converter matrix material. As described further below, aspects of the invention include processes to conformally coat the matrix—even complex matrix shapes like wool—with catalyst, thereby enabling the low loss converters described herein.

The Pt or other catalyst is typically deposited to a thickness of between about 5-20 nm, or more particularly 10-15 nm, thicknesses at which the catalyst does not affect sample flow rate (e.g., 10 nm on a 10 um fiber).

In one embodiment of a converter configured for breath analysis, a small amount of Pt is deposited on quartz wool, and is loosely packed into one or more suitable enclosures (e.g., an inert tube such as quartz tube, borosilicate glass, PTFE, or the like). In one example, about 0.2-0.4 g total weight of wool plus Pt was disposed in a tube of about 10″ length and ⅛-¼″ inner diameter. The wool was arranged to take up about 1-2″ of length in the tube, and positioned about ¼ of the way from one end. Alternatively, a different matrix, e.g. mesh, made of quartz or SiO2 can be used as substrate.

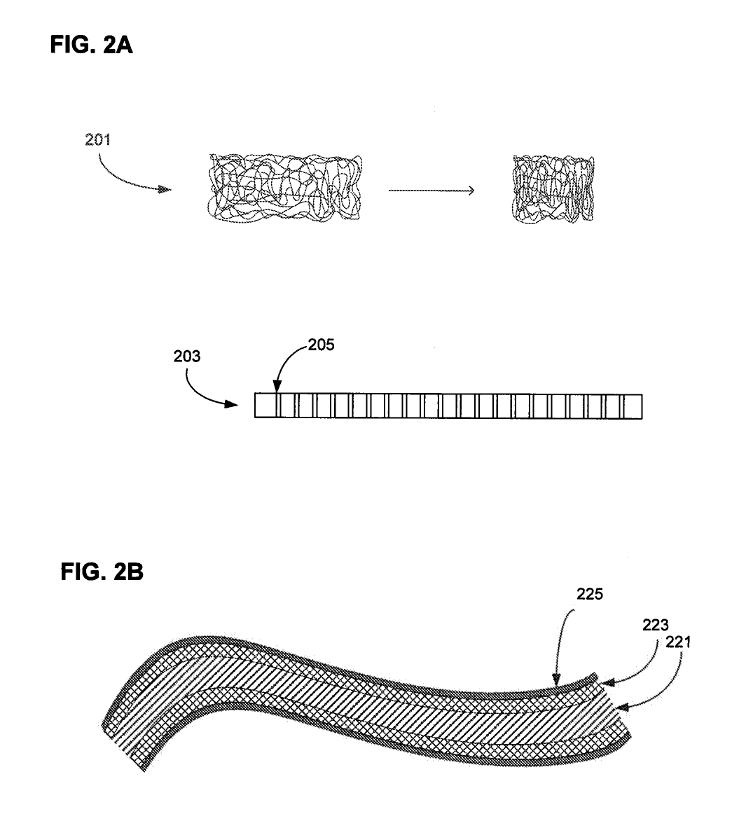

In certain embodiments, the matrix may be a shapeable material, like wool, on which the catalyst is deposited before being or packed or formed into the converter conduit. In other embodiments, the matrix is a structure that is fixed at normal operating temperatures. FIG. 2A shows examples of various forms a matrix or carrier material may take: at 201, a fibrous structure is shown that may be compacted (as in the figure), expanded or otherwise formed to fit into the converter housing. At 203, a fixed structure is shown, in this case, a wafer or other substrate 203 having microchannels 205 conformally coated with catalyst. As discussed further below, in certain embodiments, the catalyst is deposited directly into the microchannels, e.g., by atomic layer deposition. The substrate having microchannels may be connected to or part of the converter housing prior to deposition, simplifying the manufacturing process. Other examples of matrix structures include, but are not limited to, channel-containing disks, grids, meshes, honeycombs, tubes, and any other structure that provides the requisite surface area of gas flow path for the application.

In certain embodiments, the matrix is coated or otherwise modified to improve it as a surface for catalyst deposition, e.g., by reducing porosity and defects. FIG. 2B illustrates a portion of a single fiber in a fibrous matrix in which an interlayer 223 is disposed between fiber 221 and the catalyst 225. (Note that the figure is for illustrative purposes and is not drawn to scale. As indicated above, fibers are typically micron-scale, and catalyst layers nanometer-scale). For example in one embodiment, glass wool is coated with zirconia prior to catalyst deposition. Uncoated, glass wool is porous and highly reactive to NO2. The coating surfaces prepared by, e.g. atomic layer deposition, allow us to produce an inert surface, while maintaining the high surface areas. According to various embodiments, the surface is modified with a material such as zirconia, alumina, silicon nitride or silicon dioxide.

The interlayer may also be used to provide hydrophobic characteristics. Certain applications require continuous operation in humid environments. In certain embodiments, an interlayer having hydrophobic characteristics is provided. Examples of interlayer materials having hydrophobic characteristics include zirconia, alumina, silicon nitride, etc. (If sufficient to support the catalyst, these materials may be used as the matrix material itself in certain embodiments). As discussed below, a thermally conductive interlayer may also be used as a heating element. In certain embodiments, a plurality of interlayers may be disposed between the matrix and the catalyst, e.g., a metallic interlayer on the matrix to provide heating and a second interlayer on the metallic interlayer to provide a smooth, non-porous deposition surface.

### Electrical Connection to the Catalyst

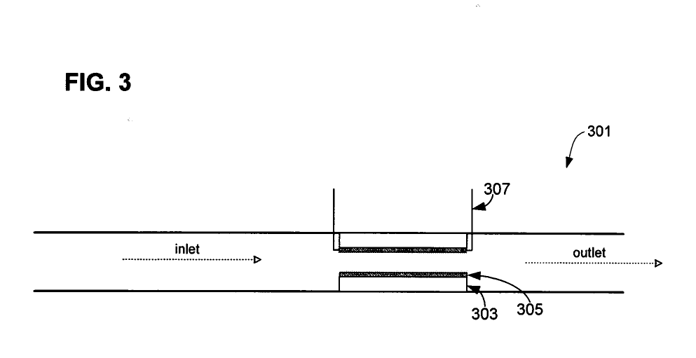

In certain embodiments, the catalytic converter includes an electrical connection to the catalyst. Unlike in conventional catalytic converters, the methods described herein permit deposition of continuous and uniform catalyst films. All particles are connected, allowing an outside electrical connection to be made to the catalyst. A cross-sectional illustration of a catalytic converter 301 having an electric connection is shown in FIG. 3. Catalyst support 303, e.g., made out of quartz, supports a continuous film of Pt or other catalyst 305. Electrical contacts 307 provide a connection to the catalyst. The electrical connection may be used to monitor electrical conductance across the catalyst, which can be used as an indicator of catalyst cleanliness and converter performance. In addition, the connection allows the electrical, rather than thermal, regeneration of the catalyst.

Note that the catalytic converter in FIG. 3 shows a catalyst support having a tube configuration with a continuous layer of catalyst deposited on the inside of the tube. Catalyst in any configuration may be electrically connected as in FIG. 3, as long as there is a continuous catalyst film. Fixed structures, such as that in FIG. 2A at 203, are particularly useful for maintaining an electrical connection. In certain embodiments, multiple connections may be made if there are electrically isolated regions of catalyst. For example, an array of tubes, each of which contains a continuous film layer of catalyst isolated from catalyst in other tubes, may include electrical connections between the catalyst layers or each catalyst layer may have an individual connection to an external electrical source.

### Heating Element

Optionally, the conversion device may include a temperature regulation mechanism, so that operating temperature may be maintained to best results. The conversion device embodiment 100 in FIG. 1 has a heating mechanism arranged adjacent the conduit and configured to maintain a selected elevated temperature of the conversion region. In one example, the heating mechanism includes a heating element 106, and a thermally conductive body 107 in effective thermal communication with the heating element and at least the conversion region 103 of the conduit 101. The conduit of the embodiment may further comprise a pre-heating region 108 disposed in sequence upstream of the conversion region and in communication with the heating mechanism, so as to provide a selected elevation in temperature of the sample during flow through the pre-heating region. The heating mechanism of the embodiment may further comprise a feed-back temperature sensor 109 and control circuitry (not shown) configured maintain a selected temperature in the conversion region. Optionally, the converter may be enclosed in an insulation material or jacket 110.

In an example, a tube is placed inside a heater with an active heated length of 6-8 inches, such that the wool is positioned at one end of the active heat area. Sample gas is introduced into the tube at the opposite end from the catalyst so it passes through the heated area first before reaching the Pt coated wool. Sample flow may be at a selected rate, e.g., between 100-500 sccm, 150-500 sccm or 200-500 sccm.

In operation, conversion of NO to NO2 within a temperature profile (e.g., the mean temperature) selected to be high enough so that conversion is sufficiently rapid, and to be low enough so that the thermodynamic properties of the gases maximize conversion (e.g., so that equilibrium rate of NO to NO2 back-conversion is minimized). In one embodiment, conversion takes place at a temperature within the range between room temperature (about 22° C.) and about 350° C., and preferably within a range of about 100° C. to about 250° C. In general higher temperatures promote fast conversion, but may be undesirable because of power, safety, and cost issues.

In the example of FIG. 1, the sample tube is enclosed in a thermally conductive body (e.g., aluminum) which contains a controllable electrical heater cartridge. Optionally, a temperature sensor (e.g., a thermistor or the like) may be disposed adjacent the tube to permit temperature of the body to be measured and used for feedback control of the cartridge. The heated tube before the catalyst section acts as a heat exchanger that warms the incoming sample gas (shown flowing left to right in the figure) to the selected conversion temperature. The catalyst (e.g., Pt on Quartz wool) provides a high surface area region where the NO gas in the sample can readily interact with the catalyst. This embodiment provides a low pressure drop as sample flows so that it does not take much energy to move the gas through the converter. The diameter of the tubing and flow rate may be selected to determine a sample exposure time within the converter (in one example, about bout 0.5-1 seconds). The inert materials ensure that the loss of NOx is minimized, and that conversion is efficient. Residence time may be significantly lower, e.g., on the order of microseconds or less. For example, for a 150 cc/min, ¼″ ID tube, 2″ converter, the residence time is less than 10 ms.

In another embodiment, the matrix itself or a thermally conductive coating thereon acts as a heating element. Relatively inert metals, such as tungsten, nickel, molybdenum, etc., may be conformally deposited on the support, such as interlayer 223 in FIG. 2B. The tungsten is then used as the heating element for the catalyst. In other embodiments, a thermally conductive matrix, e.g., made of nickel, tungsten, molybdenum, functions as the physical support for the catalyst as well as a heating element.

While the above description of the catalytic converter refers primarily to Pt catalyst and NO to NO2 converters, alternative embodiments may include additional or other catalysts (e.g., Rh, Pd and the like, and alloys and mixture thereof or with Pt). Like wise, the embodiments described may be employed to convert other gases by catalytic reaction (e.g., CO—CO2 and the like).

### 8. Methods of Fabricating Converter

Another aspect of the invention relates to novel methods of fabricating catalytic converters. As discussed above, the catalytic converters have low loss and high efficiency due to the deposition of a conformal and continuous film of catalyst nanoparticles on the matrix surface. Conventional methods of coating honeycomb structures with Pt particles typically involve wet chemistry. With wet chemistry, the reaction depends on the wetting properties making it difficult to achieve good reproducibility and size control of the nanoparticles. The methods described herein involve atomic layer deposition (ALD) of the nanoparticles. The ALD methods involves alternate doses of a Pt-containing (or other catalyst-containing) precursor and co-reactant. Unlike other types of vapor phase deposition, such as chemical vapor deposition, ALD methods are surface-controlled reactions. It has been found that ALD is an effective method of forming continuous conformal nanoparticle layers of Pt and other catalysts, even on complex surfaces such as wool or other fibrous surfaces.

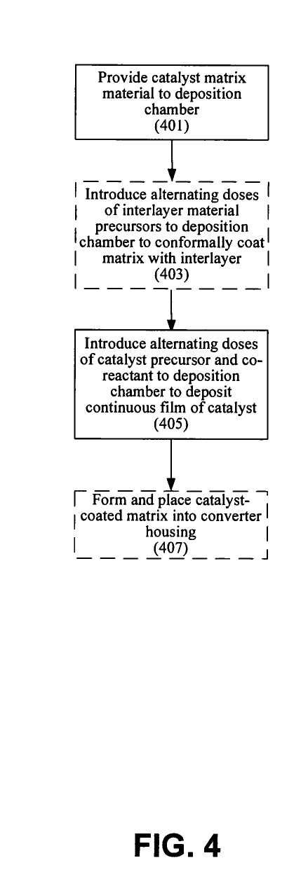

FIG. 4 shows a method of fabricating a catalytic converter according to an embodiment of the invention. First, in an operation 401, a matrix material is placed into a deposition reactor. Depending on the matrix material and the final form it will take in the converter, the material may be in this form or in another configuration. For example, a fibrous material may be placed in the reactor for deposition and later compacted or formed as needed to place into the converter. Due to the nature of the ALD process, the catalyst can be deposited directly into microchannels in silicon or quartz substrate (such as shown at 203 in FIG. 2A), enabling the matrix to be place directly into a converter housing or connected to other components in certain embodiments. Prior to or after placing the matrix material in the deposition reactor, an optional cleaning operation may be performed. Next, in an operation 403, an interlayer is deposited on the matrix by an atomic layer deposition process using alternating doses of appropriate precursors. For example, to deposit tungsten, alternate doses of a tungsten-containing precursor such as tungsten hexafluoride and a reducing agent, such as hydrogen, silane, etc., are introduced into the reactor, separated by a purge gas. Film thickness is controlled by the number of cycles, with a monolayer or less of material typically deposited in a single cycle. The catalyst layer is then deposited in an operation 405 by introducing alternating doses of a catalyst-containing precursor and a co-reactant. For example, (methylcyclopentadienyl)trimethylplatinum [MeCpPt-Me3, (CH3C5H4)Pt(CH3)3] and oxygen may be used for ALD deposition. See Aaltonen et al., Atomic Layer Deposition of Platinum Thin Films, Chem. Mater. 2003, 15, 1924-1928, incorporated by reference herein in its entirety and for all purposes. Oxygen, hydrogen or other co-reactant decomposes the ligands of the Pt-containing precursor, depositing a monolayer or less of Pt, e.g., about 0.1 nm. Thickness is controlled by the number of cycles, for example, 5-20 nm of catalyst may be deposited with 50-200 cycles. Catalyst thickness should be enough to ensure that the matrix is completely covered, with no exposed areas. Once the film is deposited, the matrix is then formed, if necessary, and placed into the converter in an operation 407.

In certain embodiments, it may be desirable to prevent deposition at certain regions of the structure placed into the ALD reactor. For example, for the structure in FIG. 2A at 203, it may be desirable to coat only the microchannels 205 and not the top or bottom surfaces of substrate 201. Similarly, if a converter matrix material is connected to components used to fit the matrix into the converter housing, deposition would not be desired on these components. Regions on which deposition is not desired can be masked or made out of a material that does not bind well to the Pt or other catalyst, e.g., with photoresist, etc.

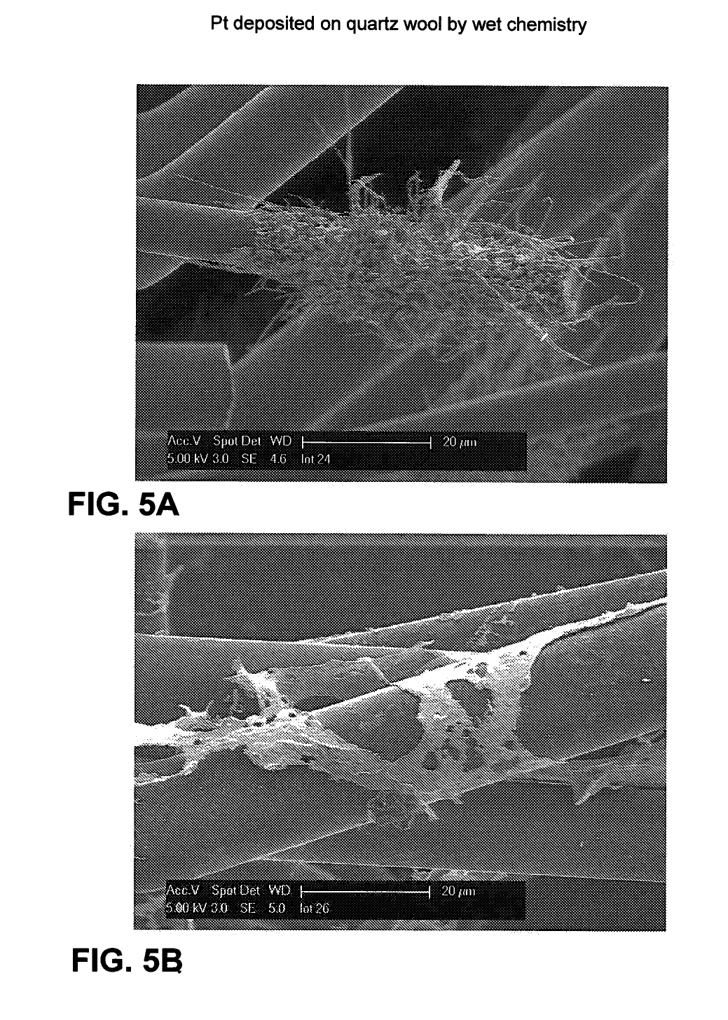

The ALD process deposits Pt nanoparticles that exhibit tight packing, with all particles connected. Because the reaction occurs on the surface, the substrate surface area is fully covered. This is in contrast to wet chemistry methods, which are difficult to control. The difference in film deposition quality is apparent from comparing images in FIGS. 5A-5D, which show Pt deposited on quartz wool by wet chemistry, to images in FIGS. 5E and 5F, which show Pt nanoparticles deposited by ALD. As can be seen from the micron-scale images in FIGS. 5A-5D, the wet chemistry process deposits Pt unevenly and leaves significant portions (micron-scale and larger gaps) of the wool uncovered. A micron-scale image in FIG. 5E shows that the ALD process deposits a continuous film on the quartz wool. The nanometer-scale image in FIG. 5F shows close packing, with minimal exposure of the wool surface. The film is continuous at nanoscale dimensions. Particle size depends on process conditions (time, temperature, etc.), with domain sizes ranging between 5 nm and 20 nm in particular embodiments, with size being quite uniform for a particular process.

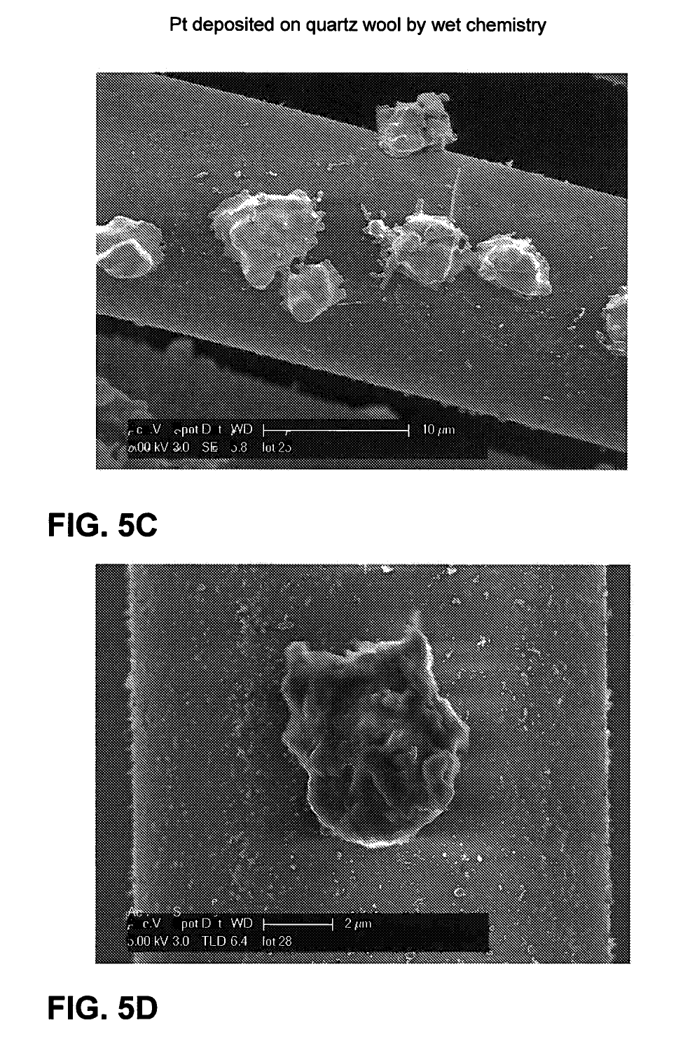

FIG. 6 is a graphical depiction of a quartz wool matrix before (601) and after (603) ALD deposition. Because ALD processes involve saturating the surface with the first reactant, and are surface-controlled, they are able to uniformly and conformally coat complex structures, such as fibrous wool, where other vapor deposition techniques including chemical vapor deposition (CVD) and physical vapor deposition (PVD) are not. With CVD, reactions take place in the vapor phase and not on the matrix surface; as a result, all of the matrix surface may not be covered. This is especially true for structures, such as that represented in FIG. 6, that require diffusion of the vapor phase components to coat the entire structure. PVD methods also are inadequate to coat such structures as deposition on any part of the structure depends on the angle of incidence and free path of the atoms. ALD methods also allow uniform distribution of the catalyst film. The critical parameter of the film for catalytic purposes is total surface area, which is chiefly controlled by particle size and distribution. Uniformly distributing the film allows good control over total surface area.

### 8. Method of Converting NO to NO2

Aspects of the invention also pertain to methods of converting NO to NO2 having low loss and high efficiency. The methods generally involve inducing a flow of a gas containing NO to an inlet portion of a converter conduit, such as depicted at 102 in FIG. 1. In certain embodiments, inducing a flow of gas may involve breathing into the inlet portion, or into a sampling portion in fluid communication with the inlet portion, in embodiments in which a breath sample is collected. In other embodiments, a pump, such as an air sampler pump used in environmental applications, may be used to induce flow through the conduit. Other methods of inducing flow into the converter applicable to the particular application may be used. In its flow through the conduit, the gas contacts a catalyst coated matrix spanning the conduit. This contact in the presence of oxygen (e.g., molecular or atomic oxygen or an oxygen-containing compound) allows a catalytic reaction of NO to NO2. (Depending on the sample source, a separate source of oxygen may or may not be needed to supply the needed oxygen.) In some cases, surface adsorbed oxygen is sufficient for the reaction to occur. The product gas is then directed toward the outlet portion, e.g., for sample collection, NO2 measurement, etc.

Flow rates depend on the specific application. For example, for breath samples, flow rates may range from 0 to 60 liters per minute. By providing a catalyst film conformally coated on a matrix high surface area, high porosity and continuous at nanoscale dimensions, the methods provide high efficiency and low loss NO to NO2 conversion, as discussed above. Another significant advantage is that the converters described above enable very short reaction residence times, allowing real time conversion. In certain embodiments, residence time is on the order of milliseconds or less, effectively providing real-time conversion. This is significant because there is no trade-off between speed and accuracy that is common to many catalytic converter systems. That is, the ALD-deposited catalysts provide short residence time, low loss and high efficiency.

### 9. Methods of Quantifying NO Including NO Breath Analysis.

In certain embodiments, NO is oxidized to form NO2 as described above, followed by detection of the resultant NO2 using a sensor configured to have a sensitivity to NO2. Aspects of the invention include methods of sensing NO in exhaled breath and in other samples, such as environmental samples. The methods involve converting the NO to NO2 as described above, and then directing the product gas stream to a NO2 analyzer to determine the presence and/or quantity of NO2 in the sample. Due to the high efficiency, low loss of the catalytic converters described above, the amount of NO in the original sample may be determined from the amount of NO2 in the product gas stream, i.e., according to various embodiments, substantially all of the NO in the sample is converted to NO2, which is present in the product gas stream (i.e., not lost due to absorption or destruction).

In one embodiment in which air is to be sampled, inhaled air may be passed through a “scrubber” device to remove environmental NO (and/or any other selected substance, such as CO2, NOx and the like) prior to administration to a patient or test subject. During or after a collection of a subsequent exhaled air sample, the sample is passed through a conversion device such as that depicted in FIG. 1 to oxidize all or a portion of the NO to NO2. Optionally, the exhaled sample may be passed through one or more filter or absorber devices to remove particulates, water vapor, atomized fluids, and/or gasses such as CO2 and the like. Similarly, in other embodiments, sensing environmental NO optionally involves scrubbing the environmental sample prior to passing it through a conversion device.

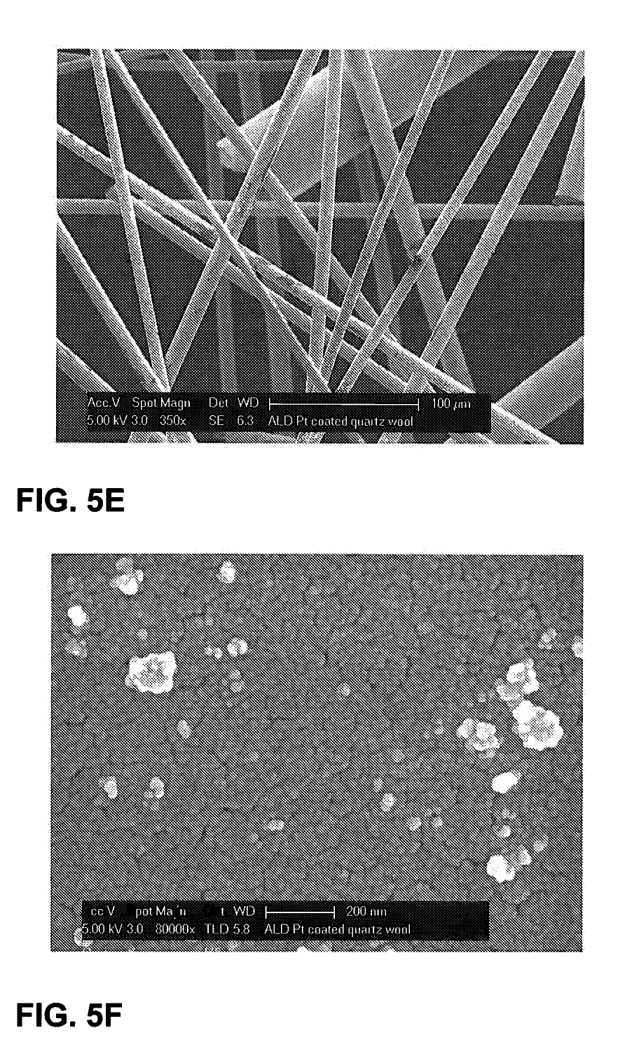

FIG. 7 illustrates the operation of one exemplary embodiment having aspects of the invention, and the effect for an electron donating (NH3) and electron withdrawing (NO2) species on a nanotube field effect transistor (NTFET) device characteristic, believed to be the result of charge transfer between the molecular species and the carbon nanotubes. In this example, the NTFET devices were fabricated using single-walled nanotubes (SWNTs) grown by chemical vapor deposition (CVD) on 200 nm of silicon dioxide on doped silicon from iron nanoparticles with methane/hydrogen gas mixture at 900° C. Electrical leads were patterned on top of the nanotubes from titanium films 35 nm thick capped with gold layers 5 nm thick, with a spacing of 0.75 μm between source and drain. The devices were contact-passivated with a liftoff-patterned SiO2 layer, which was extended over the leads and for several hundred nanometers on either side. (In other examples, the NTFET devices are fabricated using SWNTs grown by chemical vapor deposition (CVD) on 50-400 nm of silicon dioxide on doped silicon from iron nanoparticles with methane/hydrogen gas mixture at 850-950° C. Electrical leads are patterned on top of the nanotubes from titanium films 5-10 nm thick capped with gold layers 50-200 nm thick, with a spacing of 5-100 μm between source and drain.)

Upon exposure to gases, the transfer characteristics shifted left (for NH3) or right (for NO2), towards more negative or more positive gate voltages. These findings show that the exposed nanotube channel exhibits field effect transistor characteristics. Without being limited by theory, these results may be due to charge transfer between the nanotubes and the analytes involving either electron donors (NH3) or acceptors (NO2).

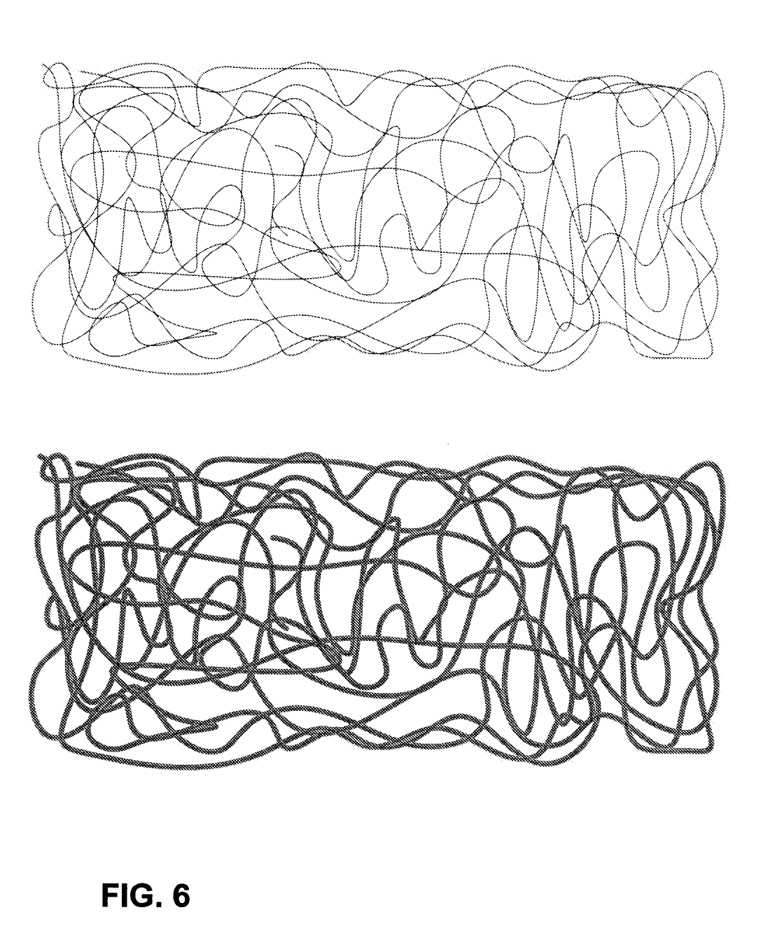

In an alternative exemplary embodiment having aspects of the invention, a nanotube device such as shown and described with in U.S. Ser. No. 11/924,328 may be employed, wherein the nanotube network may be coated with a thin polymer layer, such as poly(ethylene imine) (“PEI”). For example, the polymer layer may be about 10 nm thick. In this configuration, the device may be operated as an n-type FET. FIG. 8 shows the response of a PEI polymer-coated NTFET to four brief exposures of NO₂ gas with different concentration.

It may also be operated in a resistive mode as a sensor, and exhibits an improved response to NH3, NO2, and H2. As an example, functionalization of nanostructure devices by coating with PEI has been found to improve the response of the devices for some gases, such as NH3 and NO2, and induce a response to other gases, such as H2.

For further description, see (a) U.S. Ser. No. 10/656,898 filed Sep. 5, 2003 (published 2005-0279,987), entitled “Polymer Recognition Layers For Nanostructure Sensor Devices”; (b) A. Star, K. Bradley, J.-C. P. Gabriel, G. Grüner, “Nano-Electronic Sensors: Chemical Detection Using Carbon Nanotubes”, Pol. Mater.: Sci. Eng. 89, pp 204 (2003); and (c) U.S. Ser. No. 11/259,414 filed Oct. 25, 2005 (published 2006-0228723) entitled “Systems And Method For Electronic Detection Of Biomolecules”; each of which is incorporated by reference.

As noted above, NO measurement in breath is an important indicator of inflammatory conditions, immune response, and a number of other conditions. In particular, exhaled nitric oxide (NO) has the potential to be an important diagnostic and management indicator for airway diseases and in particular bronchial asthma. Typically, asthmatic patients have high exhaled NO levels as compared to non-asthmatic persons, and the administration of effective anti-inflammatory therapy has been correlated with a significant decrease in these NO levels.

Although existing tests of exhaled NO employing expensive, bulky and complex equipment may aid in the diagnosis and assessment of current asthma status in a clinical outpatient setting, what is needed is an inexpensive, truly portable, and patient operable NO monitoring unit to provide typical asthma patients (or their parents or caretakers) with a real-time index of the need for self-administered medication, or response to such therapy. Prompt compliance with a treatment program tailored to the patient's day-to-day (or shorter time scale) status of bronchial inflammation can prevent an asthmatic episode from becoming an emergency matter. In addition, accurate proactive control of chronic inflammatory airway conditions without over-medication can reduce cumulative tissue damage and improve long term patient outcomes.

See, for example, (a) S. A. Kharitonov et al, “Increased nitric oxide in exhaled air of asthmatic patients”, The Lancet (1994) vol. 343, pp. 133-135; (b) B. Kimberly et al, “Nasal Contribution to Exhaled Nitric Oxide at Rest and during Breathholding in Humans”, Am. J. Resp. Critical Care Med. (1996) 153 pp. 829-836; (c) A. F. Massaro et al, “Expired nitric oxide levels during treatment of acute asthma”, Am. J. Resp. Critical Care Med. (1995) vol. 152, No. 2, pp. 800-803; and (d) P. E. Silkoff et al, “Airway nitric oxide diffusion in asthma: Role in pulmonary function and bronchial responsiveness”, Am. J. Resp. Critical Care Med. (2000) 161 pp. 1218-1228; each of which publication is incorporated by reference. See also the methodology described in U.S. Pat. No. 5,447,165; No. 5,922,610; and No. 6,038,913; each of which is incorporated by reference.

Unlike CO2, which is a major component of exhaled breath (typically 1-5%), NO is generally present in only trace amounts, typically in an order of magnitude of a few parts-per-billion (ppb). For example, a non-asthmatic patient may be test for eNO in the range of 5-25 ppb, while an asthmatic patient may test in the 30-100+ ppb range. Of course measurement at these levels requires much greater detector sensitivity than for CO2. But importantly, NO is produced by metabolic processes in many different tissues and cellular responses, which are not negligible, given that trace amounts are medically relevant. In respiration, NO is produced not only in the bronchial airway, and by alveolar gas exchange from the blood, but is also produce in nasal, mouth, tracheal and throat tissue. In addition, NOx of atmospheric and localized air pollution can contribute to measurements. Therefore, substantial work has been done in the attempt to assure that the NO in sampled breath is representative of bronchial airway sources, while minimizing alternative contributions. For example, intake filters may be employed to remove ambient NO from inspired air. Techniques may be employed to exclude air emerging from the nasal cavity via the nasopharynx from the sample. In addition, exhaled NO concentrations depend substantially on expiratory flow rate.

Sample collection may include discarding an initial portion of an exhalation, followed by collecting sample air during a period of exhalation against a flow resistance or back pressure. See for example, (a) P. Silkoff et al., “Marked Flow-dependence of Exhaled Nitric Oxide Using a New Technique to Exclude Nasal Nitric Oxide”, Am. J. Respir. Crit. Care Med., (1997) 155 pp. 260-67; (b) U.S. Pat. Nos. 5,795,787 and 6,010,459, each entitled “Method and apparatus for the measurement of exhaled nitric oxide in humans”; (c) U.S. Pat. No. 6,067,983 entitled “Method and apparatus for controlled flow sampling from the airway”; (d) U.S. Pat. No. 6,733,463 entitled “Method and measuring equipment for measuring nitric oxide concentration in exhaled air”; and (e) US Published Application No. 2004-0017,570 entitled “Device and system for the quantification of breath gases”; each of which publication and patent is incorporated by reference.

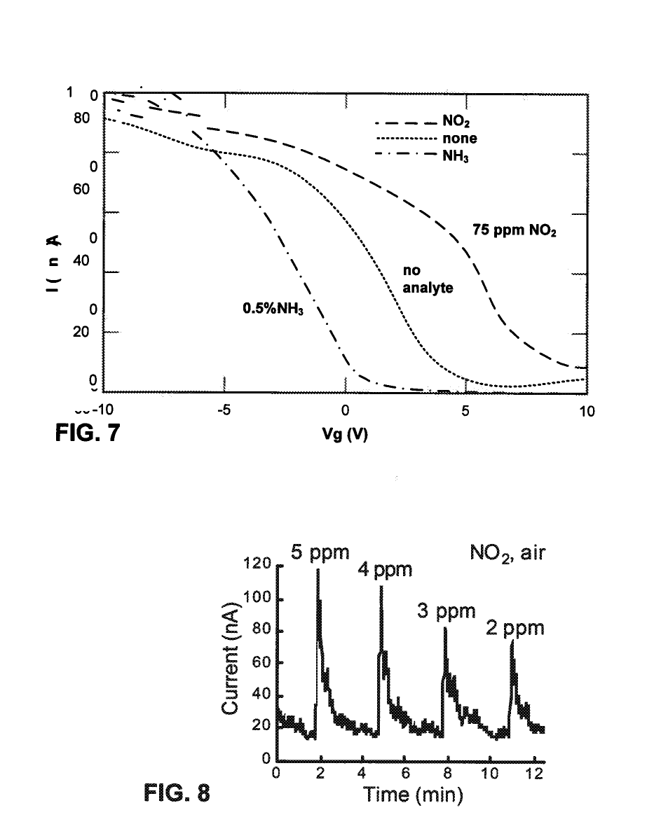

FIG. 9 is a plot showing the dependence of breath NO concentration on the exhalation rate (from the above noted U.S. Pat. No. 6,733,463), comparing healthy patients with patients with airway disease conditions. For all sets of patients, there is a marked, nonlinear reduction in concentration as exhalation rate increases. Given this strong dependence, it is desirable that the exhalation rate be systematically controlled during the measurement process, to give reproducible results which are representative of the airway condition, rather than representative of the degree of patient effort or compliance with instructions. It can also be seen in FIG. 9 that although the proportionate effect of exhalation rate on concentration is generally the same for each patient population, the absolute differences in patient population (in ppb) are greatest at the lowest exhalation rate.

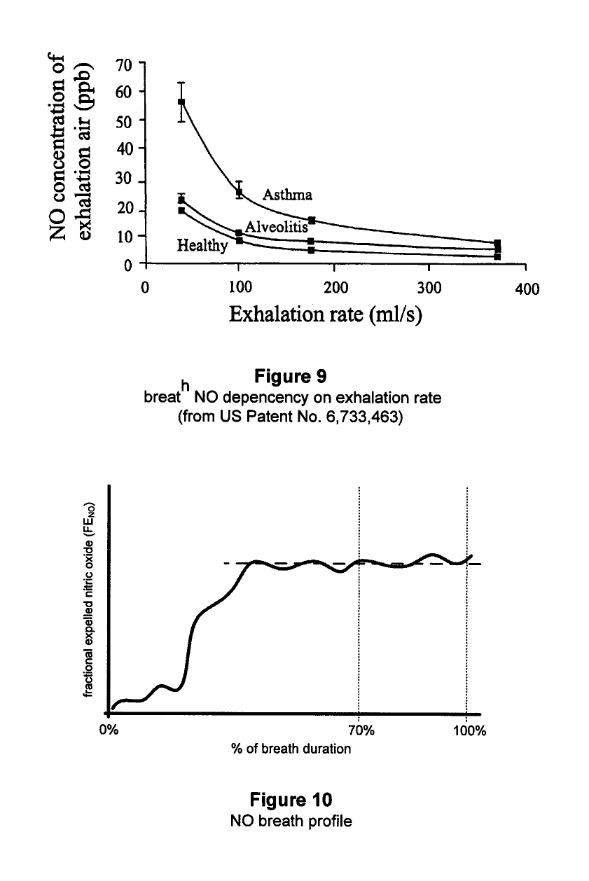

FIG. 10 is a plot showing the concentration of exhaled breath NO as a function of time or breath duration. It should be recalled that unlike CO2 (which in exhaled breath is almost entirely for alveolar source), NO in exhaled breath can be supplied as a significant fraction from a number of tissues, so that the profile, such as FIG. 10, varies with sampling factors and flow rate.

### CONCLUSION

Having thus described preferred embodiments of the methods and devices having aspects of the invention, it should be apparent to those skilled in the art that there are certain advantages to this system. It should also be appreciated that various modifications, adaptations, and alternative embodiments thereof may be made within the scope and spirit of the present invention. For example, the methods and devices described may be employed for catalytic conversion of other gases using Pt, Pd, Rh or other catalysts that may be deposited by atomic layer deposition methods or other methods that can create conformal coating on substrate support.

1. A device for converting NO to NO2 in a sample for subsequent measurement, the device comprising:
(a) a conduit including in communicating sequence: (i) an inlet portion configured to receive the sample under an input pressure sufficient to induce flow in the conduit, (ii) a conversion portion, and (iii) an outlet portion configured to dispense the sample following conversion; and
(b) a conversion material disposed within the conversion portion, the conversion material comprising a carrier material conformally coated with a packed nanoparticulate catalyst film, said catalyst film continuous at nanoscale dimensions.

2. The device for converting of claim 1, wherein the carrier material comprises a fibrous material having a high surface/mass ratio.

3. The device for converting of claim 3, wherein the carrier material comprises a quantity of quartz wool.

4. The device for converting of claim 3, wherein the catalyst comprises a metal having catalytic activity for oxidizing NO in the presence of O2 or atomic oxygen to form NO2.

5. The device for converting of claim 4, wherein the metal comprises one or more of Pt, Rh, and Pd.

6. The device for converting of claim 5, wherein the metal is configured as particles having a mean size of between 5 and 20 nm.

7. The device for converting of claim 1, further comprising:
a heating mechanism arranged adjacent the conduit and configured to maintain a selected elevated temperature of the conversion region;
wherein the heating mechanism including a heating element, and a thermally conductive body in effective thermal communication with the heating element and at least the conversion region of the conduit.

8. The device for converting of claim 7, wherein the conduit further includes a pre-heating region disposed in sequence upstream of the conversion region and in communication with the heating mechanism, so as to provide a selected elevation in temperature of the sample during flow through the pre-heating region.

9. The device for converting of claim 7, wherein the heating mechanism further comprises a feed-back temperature sensor and control circuitry configured to maintain a selected temperature in the conversion region of between about 100° C. and about 350° C.

10. The device for converting of claim 1 wherein the carrier material comprises a first material conformally coated with a second thin film material.

11. The device for converting of claim 10 wherein the first material is a fibrous material.

12. The device for converting of claim 10 wherein the second material is an alumina or a zirconia material.

13. The device for converting of claim 10 wherein the second material is a metallic material.

14. The device for converting of claim 10 wherein the second material is a hydrophobic material.

15. The device for converting of claim 1 further comprising a heating element, wherein the heating element comprises the carrier material or an interlayer disposed between the catalyst film and the carrier material.

16. The device for converting of claim 1 wherein the carrier material comprises metal wire electrically connected to an external circuit.

17. The device for converting of claim 1 further comprising one or more electrical contacts to the catalyst material.

18. The device for converting of claim 1 wherein the carrier material comprises microchannels in a substrate and the catalyst material conformally coats said microchannels.

19. The device for converting of claim 1 wherein the catalyst film is continuously electrically conductive.

20. A method for converting NO to NO2, said method comprising:
inducing flow of a gas comprising NO into a conduit, said conduit comprising a conversion portion and a conversion material disposed within the conversion portion, said conversion material comprising a carrier material conformally coated with a packed nanoparticulate catalyst film continuous at nanoscale dimensions;
allowing the flow of gas to contact the conversion material in the presence of oxygen to thereby convert at least some of the NO to NO2;
following conversion, directing the flow of gas to an outlet portion of the conduit to receive said flow.

21-41. (canceled)

42. A method of detecting NO in a gaseous sample, comprising:
directing the sample of gas to a catalytic converter in a feed gas stream, said catalytic converter comprising a conversion portion and a conversion material disposed within the conversion portion, said conversion material comprising a carrier material conformally coated with a packed nanoparticulate catalyst film continuous at nanoscale dimensions;
allowing the flow of gas to contact the conversion material in the presence of oxygen to thereby convert at least some of the NO to NO2;
following conversion, directing the product gas stream to a NO2 sensor to detect the presence or quantity of the NO2;
detecting the presence or quantity of NO2 in the product gas stream; and
determining the presence or quantity of NO in the sample based on the presence or quantity of NO2 in the product gas stream.

43-70. (canceled)

71. An NO sensor comprising:
a conduit including in communicating sequence: (i) an inlet portion configured to receive the sample under an input pressure sufficient to induce flow in the conduit, (ii) a conversion portion configured to convert NO in the sample to NO2; and (iii) an outlet portion configured to direct the sample to a sensor portion following conversion;
a conversion material disposed within the conversion portion, the conversion material comprising a carrier material conformally coated with a packed nanoparticulate catalyst film, said catalyst film continuous at nanoscale dimensions; and
the sensor portion in fluid communication with the outlet portion and comprising a sensor configured to detect or quantify the amount of NO2 in the sample.

72. (canceled)
